(19) United States
(12) Patent Application Publication
(10) Pub. No.: US 2023/0172485 A1
(43) Pub. Date: Jun. 8, 2023

(54) Breath Microbe Collection Assembly

(71) Applicant: Luke Capital Investments, LLC, Harrison Township, MI (US)

(72) Inventor: Thomas P. Luke, Harrison Township, MI (US)

(73) Assignee: Luke Capital Investments, LLC, Harrison Township, MI (US)

(21) Appl. No.: 17/924,477

(22) PCT Filed: May 12, 2021
(86) PCT No.: PCT/US2021/032037

§ 371 Date: Nov. 10, 2022

Related U.S. Application Data
(60) Provisional application No. 63/101,698, filed on May 12, 2020.

(51) Int. Cl. A61B 5/097; G01N 1/22

(52) U.S. Cl. CPC A61B 5/097; G01N 1/2205; A61B 2010/0087

(57) ABSTRACT

This disclosure provides a breath microbe collection assembly. The assembly receives both an upper respiratory breath and a lower respiratory breath of a user. The assembly includes a housing having a first chamber and a second chamber. A portion of the first chamber is configured to receive the upper respiratory breath and the lower respiratory breath. The second chamber is configured to receive the lower respiratory breath. A stop is mounted within the first chamber. A valve is mounted to the housing and has a pivoting part and a diverting part. The pivoting part is adjacent one side of the stop when the valve is in an initial position. The diverting part is adjacent another side opposite the one side of the stop when the valve is in an activated position. The valve automatically returns to the initial position when a volume of an initial breath of said user is dissipated.

The present application claims priority to and all the benefits of U.S. Provisional Application No. 63/101,698, filed May 12, 2020, which is hereby expressly incorporated by reference in its entirety.

### FIELD OF THE DISCLOSURE

This disclosure generally relates to a microbe collection device. More specifically, this disclosure relates to a breath microbe assembly including a housing, a stop, a block, and a valve having a pivoting part and a diverting part.

### BACKGROUND

Respiratory infections are a leading cause of death, however, identifying the microbial nature of the infections requires the use of an invasive or unpleasant collection process that could endanger health care workers. There is a need for a simple, cost-effective method for obtaining respiratory viral particles. Often respiratory infectious particles are collected through expectorated sputum. However, not all patients are able to spontaneously generate sputa such as HIV patients, most of those infected with bacterial pneumonia, very ill patients, and children. In cases for which sputum can't be obtained, more invasive techniques such as bronchoalveolar lavage is used to collect samples from the lower respiratory tract. Instead of lavage, capturing the particles through a breath device is preferable because it is less invasive, can be done anywhere (home or office), and does not require medically trained personnel. There is a need for the ability of a patient to submit respiratory viral and bacterial samples for analysis via a breathing apparatus that differentiates upper and lower airway samples. Furthermore, there is a need for a device that differentiates microbes coming from the upper and lower respiratory tract that further aids diagnosis.

### SUMMARY OF THE DISCLOSURE AND ADVANTAGES

This disclosure provides a breath microbe collection assembly. The assembly receives both an upper respiratory breath and a lower respiratory breath of a user. The assembly includes a housing having a first chamber and a second chamber. A portion of the first chamber is configured to receive the upper respiratory breath and the lower respiratory breath. The second chamber is configured to receive the lower respiratory breath. A stop is mounted within the first chamber. A valve is mounted to the housing and has a pivoting part and a diverting part. The pivoting part is adjacent one side of the stop when the valve is in an initial position. The diverting part is adjacent another side opposite the one side of the stop when the valve is in an activated position. The valve automatically returns to the initial position when a volume of an initial breath of said user is dissipated.

This disclosure also provides a collection system. The collection system includes a housing having a first chamber and a second chamber. A portion of the first chamber is configured to receive an upper respiratory breath from a user and a lower respiratory breath from the user. The second chamber is configured to receive the lower respiratory breath. A valve is mounted to the housing and has a pivoting part and a diverting part with the valve movable between initial and activated positions to allow a breath into a first collection device, and then to divert the breath into a second collection device when the valve is in the activated position. A first collection device has a first open end and a first vented end and is configured to collect an upper respiratory breath microbe sample from the user. A second collection device having a second open end and a second vented end and configured to collected a lower upper respiratory breath microbe sample from the user. The first collection device is mounted to the first chamber and the second collection device is mounted to the second chamber.

This disclosure also provides a method of using a collection system. The method includes blowing a breath containing microbes into a first chamber of a breath microbe assembly. The method also includes collecting microbes of an upper respiratory breath in a first collection device, pivoting a valve in the breath microbe assembly to direct breath containing microbes to a second collection device, and collecting microbes of a lower respiratory breath in a second collection device. The method also includes removing the first collection device after collecting the microbes of the upper respiratory breath, mounting a first cap on a first open end of the first collection device, and mounting a second cap on a first vented end of the first collection device. The method also includes removing the second collection device after collecting the microbes of the lower respiratory breath, mounting a third cap on a second open end of the second collection device, and mounting a fourth cap on a second vented end of the second collection device.

This disclosure provides a device that differentiates microbes coming from the upper and lower respiratory tract of a user and that further aids diagnosis. This device collects and separates microbes (bacterial and viral) coming from the upper and lower respiratory tracts, then concentrates the microbes so that they may be inactivated, stabilized and/or visualized for further analysis.

### BRIEF DESCRIPTION OF THE FIGURES

Other advantages of the present invention will be readily appreciated, as the same becomes better understood by reference to the following detailed description when considered in connection with the accompanying drawings wherein:

FIG. 1 shows a microbe collection assembly after use by a user, such as a patient, according to an embodiment of the disclosure where second and fourth caps are mounted to the first and second chambers during a transport mode;

FIG. 2 is a view of a user as the user is about to use a microbe collection assembly according to an embodiment of the disclosure;

FIG. 3 is a side view of the embodiment of FIG. 1;

FIG. 4 is a perspective view of the embodiment of FIG. 1;

FIG. 5 is a front view of the embodiment of FIG. 1;

FIG. 6 is a bottom view of the embodiment of FIG. 1;

FIG. 7 is a perspective view of the embodiment of FIG. 1 without the second cap;

FIG. 8 shows an embodiment of the disclosure before a mouthpiece, first and second collection devices, second and forth caps are mounted to a housing including first and second chambers;

FIG. 9 shows a valve, a stop and a block inside a housing according to one embodiment of the disclosure where the valve includes a pivoting part and a diverting part and is in an initial position;

FIG. 10 shows the embodiment of FIG. 9 before a mouthpiece, first and second collection devices, second and forth caps are mounted to a housing including first and second chambers;

FIG. 11 shows a valve, a stop and a block inside a housing according to one embodiment of the disclosure where the valve includes a pivoting part and a diverting part and is in an activated position;

FIG. 12 is a perspective view of FIG. 11;

FIG. 13 shows either a first collection device or a second collection device where caps are mounted on each end of the device according to one embodiment;

FIG. 14 is a view of FIG. 13 before caps are mounted;

FIG. 15 is a cross-sectional view of FIG. 13;

FIG. 16 shows either a first cap or a third cap according to one embodiment of the disclosure; and

FIG. 17 shows either a second cap or a fourth cap according to one embodiment of the disclosure.

### DETAILED DESCRIPTION OF THE DISCLOSURE

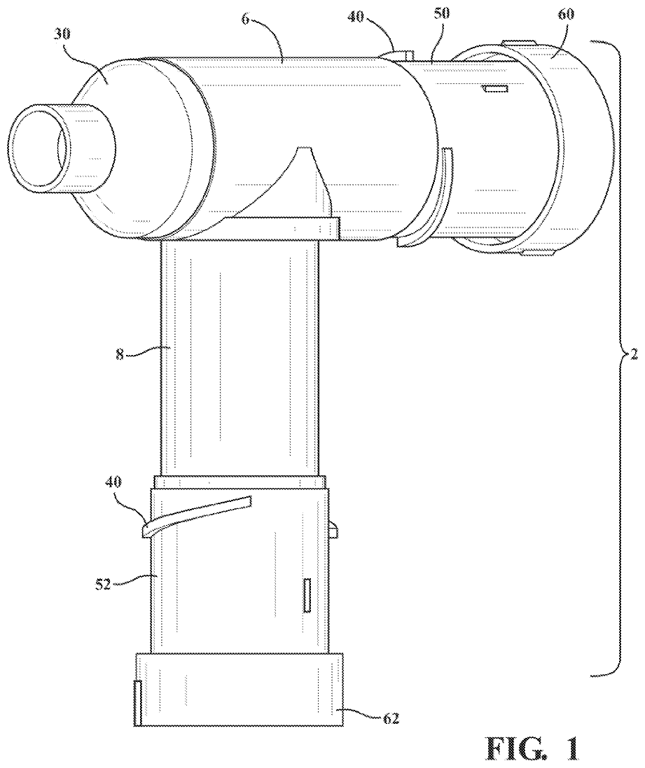

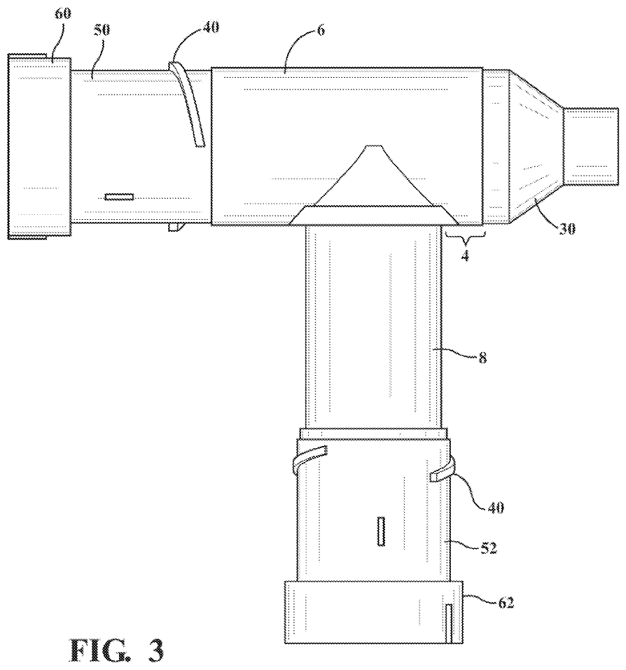

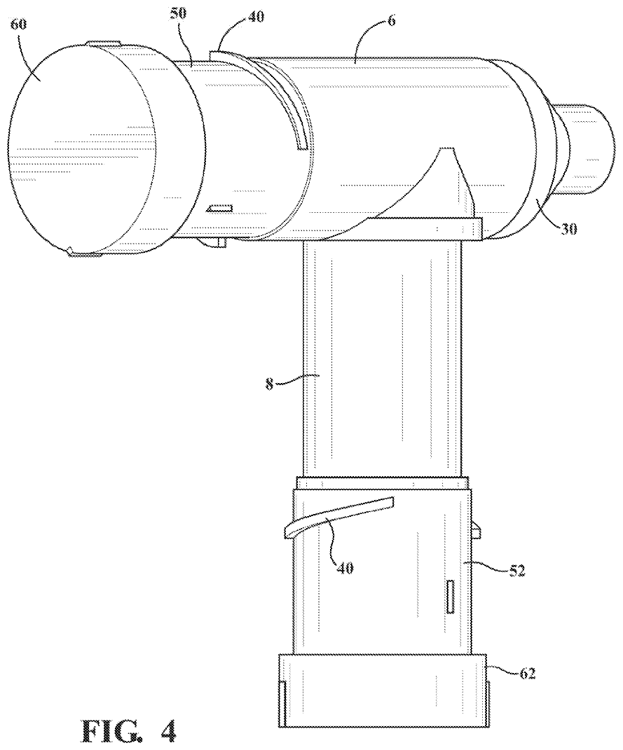

Referring to FIG. 1, wherein like numerals indicate corresponding parts throughout the figures, a breath microbe collection assembly 2 is shown. The assembly 2 receives both an upper respiratory breath and a lower respiratory breath of a user. Upper respiratory breath is breath from the upper respiratory tract of a patient. Lower respiratory breath is breath from the lower respiratory tract of a patient. The user may be a human patient, such as a patient that is suffering from cold or flu-like symptoms, or other symptoms that are indicative of a respiratory disease. Microbes in the breath of a patient are collected using the breath microbe collection assembly 2. In the embodiment shown in FIG. 1, the assembly 2 includes second and fourth caps 60, 62 during a transport mode. A side view of the assembly is shown in FIG. 1 and a perspective view of FIG. 3 is shown in FIG. 4. The second and fourth caps 60, 62 are not included in the assembly 2 during a mode of operation in which a user is blowing a breath of air through the assembly 2.

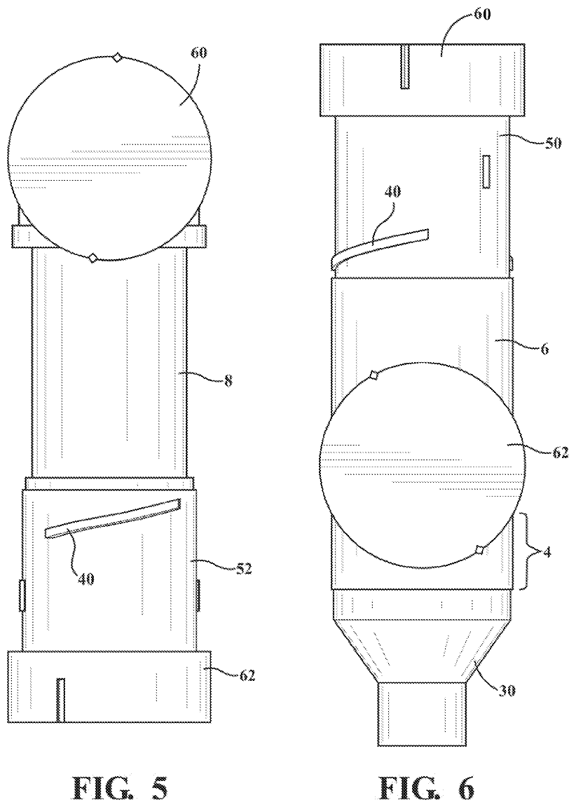

FIG. 5 is a front view of the embodiment in FIG. 1 where the second cap 60 is in front. The first chamber 6 is behind the front of the second cap 60 from this view. FIG. 6 is a bottom view of the embodiment of FIG. 1 where the fourth cap 62 is in front. The second chamber 8 is behind the front of the fourth cap 62 from this view.

The assembly 2 includes a housing having a first chamber 6 and a second chamber 8. The first chamber 6 may be any shape. For example, the first chamber 6 may have a substantially cylindrical shape or a substantially rectangular shape. In preferred embodiments, the first chamber 6 is substantially cylindrical in shape. The second chamber 8 may be any shape. For example, the second chamber 8 may have a substantially cylindrical shape or a substantially rectangular shape. In preferred embodiments, the second chamber 8 is substantially cylindrical in shape. The first and second chambers 6, 8 may form a T-shaped housing. For example, the first chamber 6 includes a hole (not shown) on a side of the first chamber 6 and the lower chamber 8 is inserted into the hole to form the T-shaped housing.

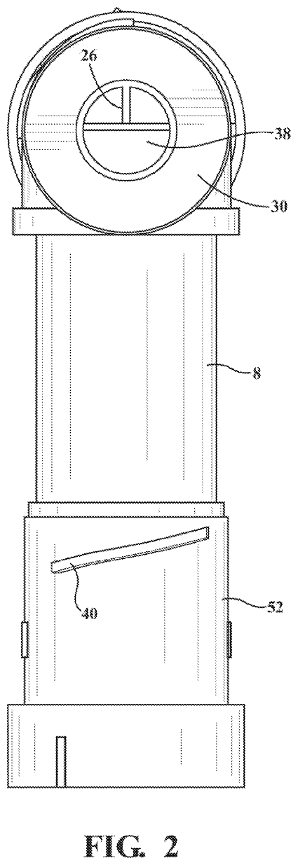

FIG. 2 is a view of a user as the user is about to use a microbe collection assembly according to an embodiment of the disclosure. In this embodiment, the assembly includes mouthpiece 30 and inclined portion 38. Looking through a hole of the mouthpiece 30, the user is able to see a front view of the included portion 38 and stop 26.

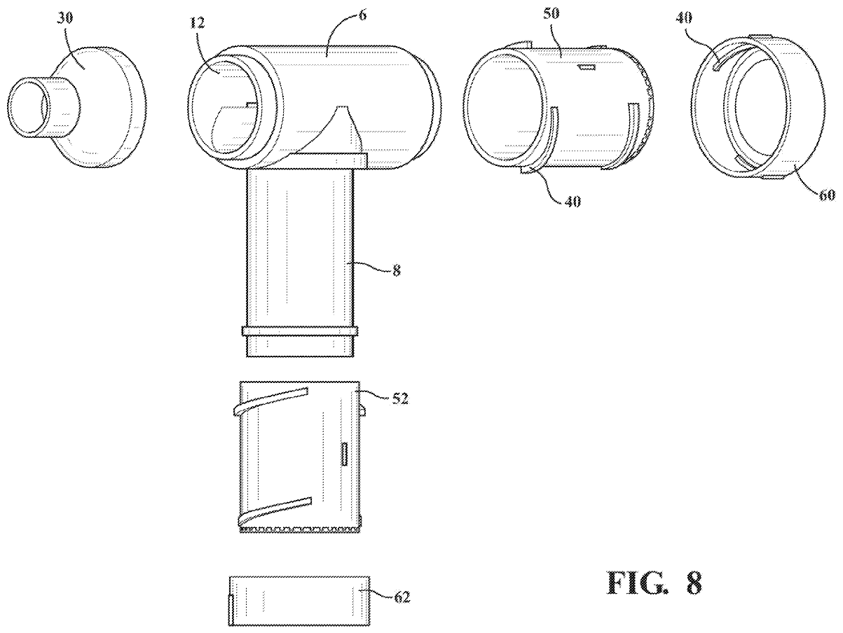

A portion 4 of the first chamber 6 is configured to receive the upper respiratory breath and the lower respiratory breath. A user, such as a patient, blows a volume of breath into one end 12 of the first chamber 6, as shown in FIG. 8. This breath includes both upper and lower respiratory breath. This end 12 of the first chamber 6 may be adjacent the portion 4 of the first chamber 6 that is configured to receive both the upper and lower respiratory breaths. As such, the portion 4 of the first chamber 6 receives both upper and lower respiratory breaths. The breath, which includes the upper respiratory breath and lower respiratory breath, also includes microbes.

The second chamber 8 is configured to receive the lower respiratory breath. As described further below, lower respiratory breath is diverted to the second chamber 6.

The first chamber 6 and second chamber 8 each include an initial volume of air. When a patient blows a volume of breath into an end 12 of the first chamber 6, the volume of breath begins to displace the initial volume of air. The volume of air continues to be displaced by the volume of breath as the breath travels through the first chamber 6 and the second chamber 8.

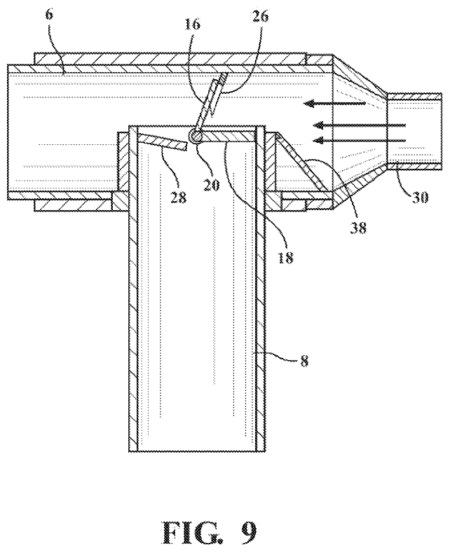

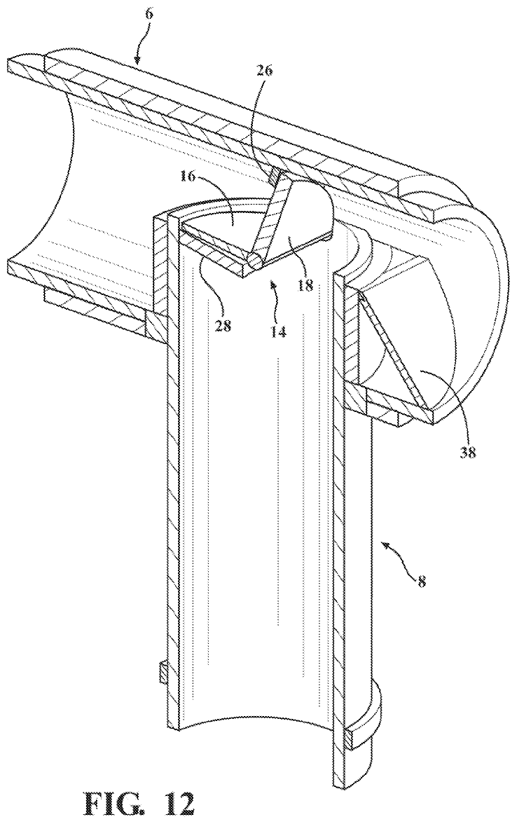

Referring to the embodiments of the disclosure shown in FIGS. 9-12, a stop 26 is mounted within the first chamber 6. In some embodiments, the stop 26 is mounted at an angle within the first chamber 6. In some preferable embodiments, the angle is not a right angle such that the stop 26 extends straight into the first chamber 6. In these preferable embodiments, the stop 26 extends at an angle into the first chamber 6. Further, in certain embodiments, the stop 26 is further defined as a slanted tab that extends into said first chamber 6 along a portion of a shape of said first chamber 6. For example, as shown in FIG. 12, if the first chamber 6 is substantially cylindrical in shape, the stop 26 extends into the first chamber 6 in a crescent shape.

A valve 14 is mounted to the housing and has a pivoting part 16 and a diverting part 18. The valve 14 may also include a pivot point 20. In these embodiments, the pivoting part 16 and the diverting part 18 each pivot about the pivot point 20 to move the valve 14 from an initial position to an activated position and then to return the valve 14 to the initial position. The valve 14 may be mounted to the first chamber 6 or the second chamber 8. In certain embodiments, the valve 14 is mounted in the second chamber 8. The valve 14 may be mounted by any means known in the art. For example, the valve 14 may be mounted in the second chamber 8 by inserting the valve into small holes (not shown) on opposing sides of the second chamber 8.

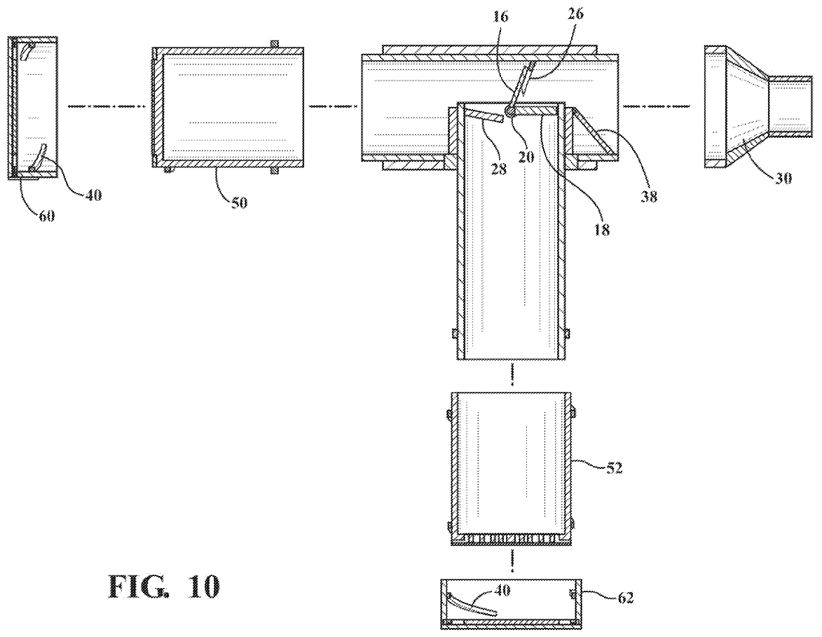

In an initial position of the valve, the pivoting part 16 of the valve 14 is adjacent one side of the stop 26, as shown in FIGS. 9 and 10. In certain embodiments, the pivoting part 16 abuts one side of the stop 26. In the initial position, the diverting part 18 lays partially across the second chamber 8 so as to close off or block the second chamber 8. In certain embodiments, the diverting part 18 is weighted compared to the pivoting part 16. This aids in maintaining the initial position of the valve 14. That is, the weighted part 18 facilitates said automatic return of the valve.

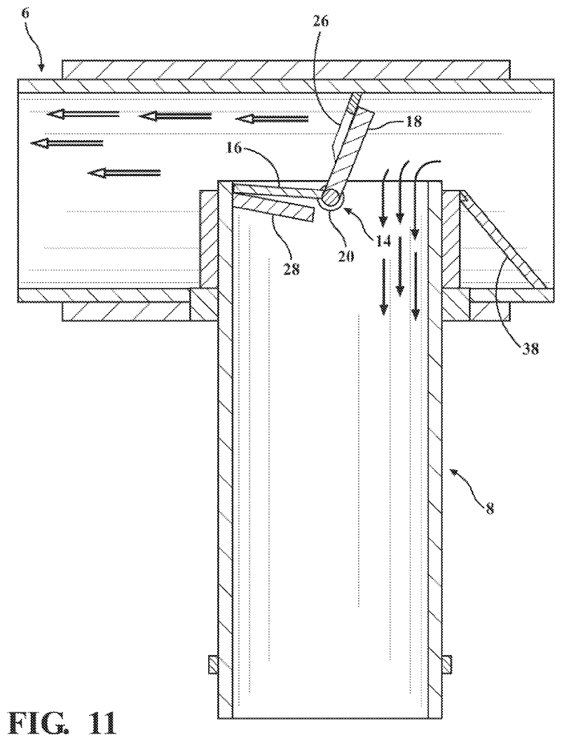

In an activated position of the valve 14, the diverting part 18 is adjacent another side opposite the one side of the stop 26, as shown in FIGS. 11 and 12. In certain embodiments, the diverting part 18 abuts the another side opposite the one side of the stop 26. The pivoting part 16 of the valve 14 is pushed back and pivots to partially block the second chamber 8. However, because the diverting part 18 is adjacent the side opposite the one side, as shown in FIGS. 11 and 12, a volume of breath is able to flow into the second chamber 8.

The valve 14 automatically returns to the initial position when the volume of breath of said user is dissipated. As described above, the first chamber 6 and second chamber 8 each include an initial volume of air. When a patient blows a volume of breath into an end 12 of the first chamber 6, the volume of breath begins to displace the initial volume of air. The volume of air continues to be displaced by the volume of breath as the breath travels through the first chamber 6 and the second chamber 8. The volume of breath contains microbes from both the upper respiratory breath and lower respiratory breath. When a volume of breath reaches the valve 14, the volume of breath has displaced the volume of air initially in the first chamber 8. An initial portion of this volume of breath pushes the pivoting part 16 of the valve 14 that is adjacent stop 26 back to place the valve 14 in the activated position. That is, when the volume of breath reaches the valve 14, the valve 14 is activated such that the pivoting part 16 of the valve is pushed back and pivots to partially block the second chamber 8, and the diverting part 18 pivots to be adjacent the side opposite the one side of the stop 26, as shown in FIGS. 11 and 12, opening up a pathway into the second chamber 8. That is, a volume of breath pushes the pivoting part to move the valve from the initial position to the activated position.

The volume of breath that pushes the pivoting part 16 is an initial volume of breath and this initial volume of breath, including microbes, travels through the remaining portion of the first chamber 6. However, the volume of breath that follows this initial volume of breath flows into the second chamber 8 because a pathway for this following volume of breath is open into the second chamber, since the diverting part 18 has pivoted and opened up a pathway into the second chamber 8. This following volume of breath, including microbes, now displaces the initial volume of air in the second chamber 8. Arrows shown in FIG. 9 show the volume of breath travelling from a mouthpiece 30 into the first chamber 6, specifically portion 4 of first chamber 6, including upper and lower respiratory breaths in these embodiments of the disclosure. Double arrows in FIG. 11 show the initial volume of breath that is the upper respiratory breath travelling through the first chamber 6 and single arrows in FIG. 11 shows the following volume of breath that is the lower respiratory breath travelling into the second chamber 8 in these embodiments of the disclosure.

The valve 14 automatically returns to the initial position when a volume of breath of the user is dissipated. In certain embodiments, when the entire volume of breath has displaced the volume of air initially in the first and second chambers 6, 8, the process of a user blowing breath into the first chamber 6 may be repeated such that further breath samples from the same user may be collected.

In some embodiments, a block 28 is disposed within the first chamber 6 or the second chamber 8. In preferable embodiments, the block 28 is disposed adjacent the valve 14 in the second chamber 8. In certain embodiments, the block 28 extends across the portion of the second chamber 8 at an angle so that the pivoting part 16 lays substantially flat on top of the block 28 when the valve 14 is in the activated position. The block 28 prevents back flow of said upper respiratory breath into said second chamber 8. The block 28 also reduces the amount of turbulence caused by the volume of breath of the user as the volume of breath travels through the first chamber 6.

In some embodiments, a mouthpiece 30 is mounted to the first chamber 6. For example, the mouthpiece 30 may be mounted to the end 12 of the first chamber 6, such that a user blows into the mouthpiece 30 and the volume of breath travels into the first chamber 6 from the mouthpiece 30. The mouthpiece 30 may have any shape suitable for directing a volume of breath from a user to the first chamber 6. For example, as shown in the Figures, the mouthpiece 30 may have a funnel shape so that the user's breath is channeled and focused into the first chamber 6.

In certain embodiments, the first chamber 6 includes an inclined portion 38 in a portion of the first chamber 6, as shown in FIG. 2. The inclined portion 38 channels the volume of breath from a user into a smaller portion of the first chamber 6. This focuses the volume of breath and forces the breath through a smaller area/portion of the first chamber 6, giving the volume of breath more force as it travels through the first chamber 6. Further, the inclined portion 38 smooths out the flow of the volume of breath and prevents turbulence that would otherwise be caused by the volume of breath.

The material that forms the first and second chambers 6, 8, the valve 14 including the pivoting part 16 and diverting part 18, and the mouthpiece 30 may be made of any material that does not attract microbes; for example, a material that is known that microbes will not stick to. This material may be ABS plastic (acrylonitrile butadiene styrene plastic), which is know to not attract microbes and/or viral particles. In these embodiments, as the microbes in the volume of breath travels through the housing including the first chamber 6 and second chamber 8, the valve 14 including the pivoting part 16 and diverting part 18, and the mouthpiece 30, the microbes are not attracted to the material that forms these components and thus can be collected in the first and second collection devices as described below.

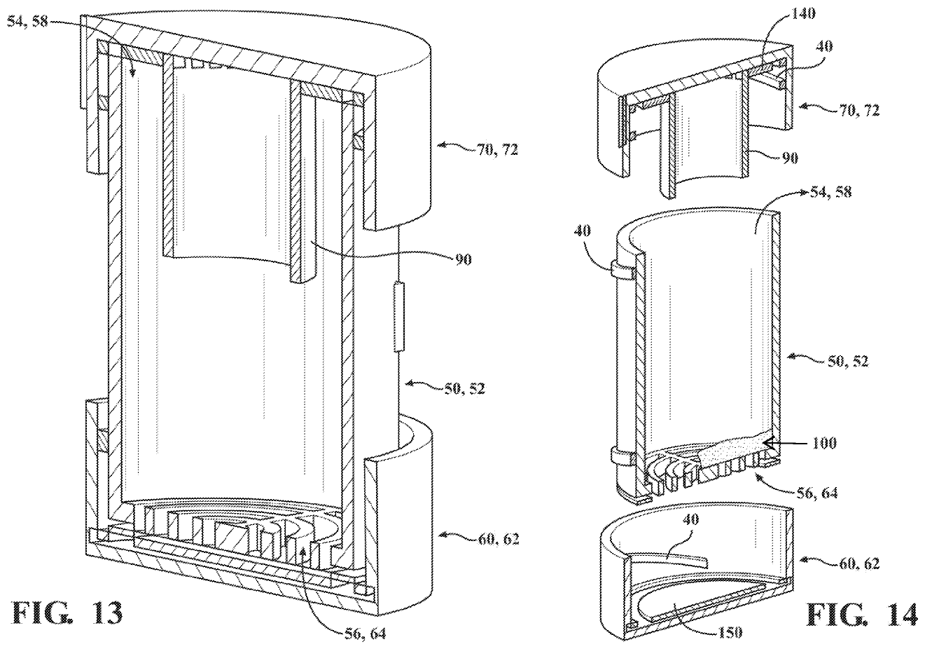

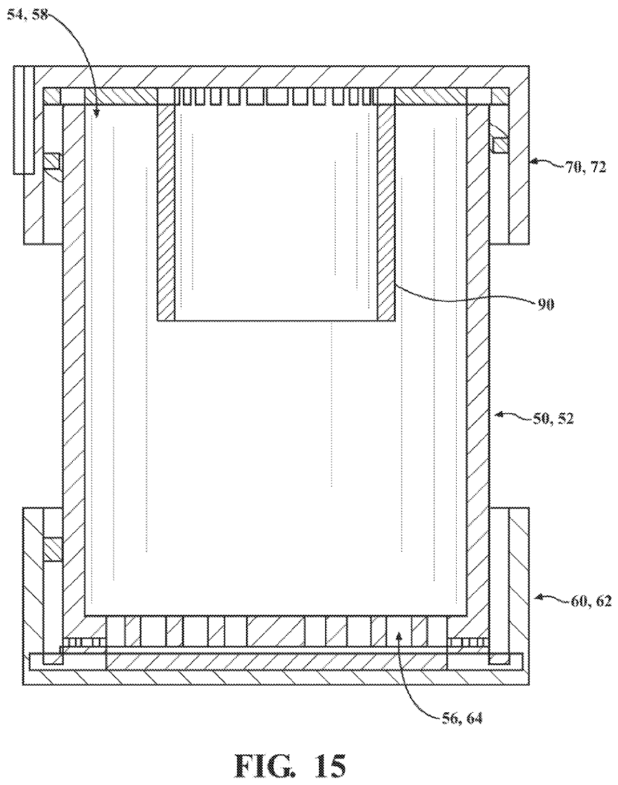

A collection system is also disclosed. Microbes that are present in the upper respiratory tract are collected in a first collection device 50. Microbes that are present in the lower respiratory tract are collected in a second collection device 52. The structure of the first collection device 50 and the second collection device 52 may be the same or different. In preferred embodiments, the structures of the first and second collection devices 50, 52 are the same, and are shown in FIGS. 13-15. The collection system includes a housing having a first chamber 6 and a second chamber 8. A valve 14 is mounted to the housing and includes a pivoting part 14 and a diverting part 16, with the valve movable between initial and activated positions to allow a breath into a first collection device, and then to direct said breath into a second collection device when the valve is in the activated position.

The collection system includes a housing having a first chamber 6 and a second chamber 8. A portion 4 of the first chamber is configured to receive an upper respiratory breath from a user and a lower respiratory breath from the user. The second chamber 8 is configured to receive the lower respiratory breath. A valve 14 is mounted to the housing and has a pivoting part 16 and a diverting part 18 with the valve movable between initial and activated positions to allow a breath into a first collection device 50, and then to divert the breath into a second collection device 52 when the valve is in the activated position. The valve being movable to allow the breath into the first collection chamber happens at any point in between the initial and activated positions. That is as the valve is pivoting about the pivot point and the pivoting part is moving away from the stop 26 and the diverting part is moving toward the stop 26, breath moves through/travels all the way through the first chamber 6 into the first collection device 50. When the valve is in the activated position, and the diverting part is adjacent the stop 26, breath is then directed into the second chamber 8 only and into the second collection device 52. The first collection device 50 may be mounted to the first chamber 6 and the second collection device 52 may be mounted to the second chamber 8.

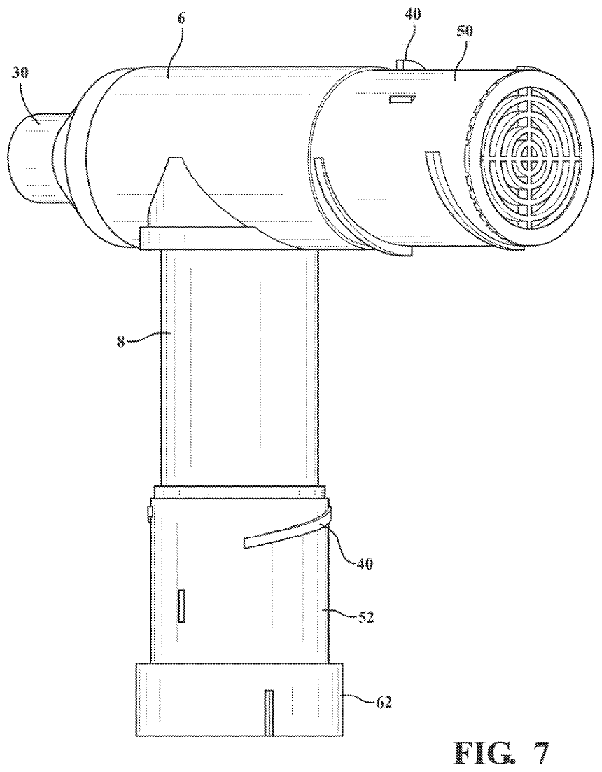

A first collection device 50 has a first open end 54 and a first vented end 56 and is configured to collect an upper respiratory breath microbe sample from said user. FIG. 7 shows the embodiment of FIG. 1 with the second cap 60 removed such that the vented end 56 of the first collection device 50 is visible.

As the initial volume of breath travels past the pivoting part 16 of the valve 14 and through the first chamber 6, the breath reaches and is collected in the first collection device 50 which is mounted to the first chamber 6. The first collection device 50 may be mounted in any suitable way. For example, the device 50 may be mounted to the first chamber 6 by the first chamber being inserted into the first open end 54 of the first collection device 50. The first collection device 50 may be made of a material that is known to bond with microbes and/or viral particles. For example, the first collection device 50 may be made of polystyrene (PS). When the volume of breath reaches the first collection device 50, the microbes are attracted to the PS material of the first collection device 50 and are therefore, collected.

In certain embodiments, the first collection device 50 may be any suitable shape that is compatible with the shape of the first chamber 6, such as a rectangular or cylindrical shape. In certain embodiments, when the first chamber 6 has a substantially cylindrical shape, the first collection device 50 also has a substantially cylindrical shape. In certain embodiments, the outer regions of the first collection device 50 includes threads 40, such that a cap may be screwed onto each end 54, 56 of the collection device 50. In other embodiments, the first collection device 50 is merely inserted into a cap on each end 54, 56 of the first collection device 50.

The first vented end 56 of the first collection device 50 may have any configuration suitable for venting breath so that there is no air build-up inside the housing including the first chamber 6 and second chamber 8. For example, the vented end 56 may have a configuration of concentric circles anchored by crossed lines, as shown in FIGS. 13-15. The vented end 56 also may support any filter paper 100 inserted into the collection device.

In certain embodiments, a filter paper disc 100 may be inserted into the first collection device 50 to be internally adjacent to the first vented end 56. The filter paper 100 may be any suitable material. For example, the filter paper 100 may be 1 micron thick standard laboratory filter paper. The filter paper 100 aids in collecting microbes in the first collection device 50.

A second collection device 52 has a second open end 58 and a second vented end 64 and is configured to collect a lower respiratory breath microbe sample from said user. As the volume of breath that follows the initial volume of breath travels into the second chamber 8 due to the activated position of the diverting part 18, the breath reaches and is collected in the second collection device 52 which is mounted to the second chamber 8. The second collection device 52 may be mounted to the second chamber 8 in any suitable way. For example, the device 52 may be mounted by the second chamber 8 being inserted into the second open end 58 of the second collection device 50. The second collection device 52 may also be made of a material that is known to bond with microbes and/or viral particles. For example, the second collection device 52 may be made of polystyrene (PS). When the volume of breath reaches the second collection device 52, the microbes are attracted to the PS material of the second collection device 52 and are therefore, collected.

In certain embodiments, the second collection device 52 may be any suitable shape that is compatible with the shape of the second chamber 8, such as a rectangular or cylindrical shape. In certain embodiments, when the second chamber 8 has a substantially cylindrical shape, the second collection device 52 also has a substantially cylindrical shape. In certain embodiments, the outer regions of the second collection device 52 includes threads 40, such that a cap may be screwed onto each end 58, 64 of the collection device 52. In other embodiments, the second collection device 50 is merely inserted into a cap 58, 64 on each end of the second collection device 52.

The second vented end 64 of the second collection device 52 may have any configuration suitable for venting breath so that there is no air build-up inside the housing including the first chamber 6 and second chamber 8. For example, the vented end 64 may have a configuration of concentric circles anchored by crossed lines, as shown in FIGS. 13-15. The vented end 64 also may support any filter paper 100 inserted into the collection device 52. The filter paper 100 used in the first collection device 50 may be the same material as the filter paper 100 used in the second collection device 52, however the same filter paper is not used in both devices 50, 52 when collecting a sample of breath.

In certain embodiments, a filter paper disc 100 may be inserted into the second collection device 52 to be internally adjacent to the second vented end 64. The filter paper 100 may be any suitable material. For example, the filter paper 100 may be 1 micron width standard laboratory filter paper. The filter paper 100 aids in collecting microbes in the second collection device 52.

In order to seal the first and second collection devices 50, 52 so that they may be transported to a place, for example a laboratory, for evaluation of the microbes collected, caps may be mounted to each end 54, 58, 56, 64 of each of the first and second collection devices 50, 52.

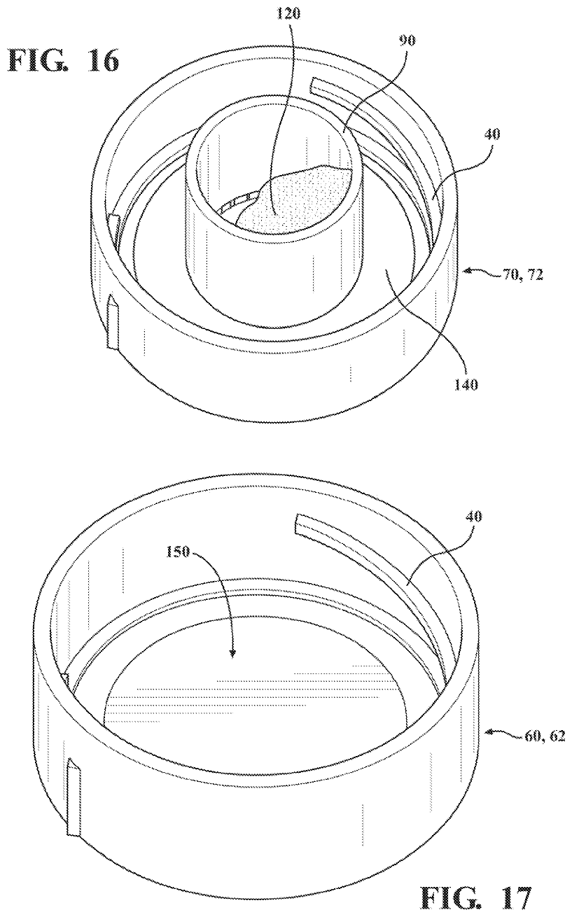

For the first collection device 50, a first cap 70 may be mounted on the first open end 54. In certain embodiments, the first cap 70 is a simple cap that may be inserted onto the first collection device 50. In other embodiments, the first cap 70 has threads 40 that may be screwed onto the first collection device 50 to seal the first open end 54. In preferred embodiments, the first cap 70 has an inner cylinder 90 taller than the first cap 70, as shown in FIG. 16. The inner cylinder 90 is configured to hold breath stabilizing media 120. For example, the breath stabilizing media 120 is a liquid, as shown in FIG. 16. In certain embodiments, the first cap 70 includes silicone material 140 surrounding the inner cylinder 90. The silicone material 140 aids in sealing in any liquid of the stabilizing media 120 and prevents leakage of the stabilizing media 120. In certain embodiments, breath stabilizing media 120 is pre-filled into the inner cylinder 90 and sealed with foil. When the first cap 70 is about to be used, the foil is removed before the first cap 70 is mounted to the first collection device 50 having microbes therein.

For the first collection device 50, a second cap 60 may be mounted on the first vented end 56. In certain embodiments, the second cap 60 is a simple cap that may be inserted onto the first collection device 50. In other embodiments, the second cap 60 has threads 40 that may be screwed onto the first collection device 50 to seal the first vented end 56, as shown in FIG. 17. In certain embodiments, the second cap 60 includes a disk of silicone material 150 inserted into the second cap 60. In these embodiments, a breath stabilizing media 120′, which is liquid, may be applied inside the first collection device 50 after the second cap 60 is mounted to the device 50. The disk of silicone material 150 aids in sealing the liquid of the stabilizing media 120′ and prevents leakage of the stabilizing media 120′.

For the second collection device, a third cap 72 may be mounted on the second open end 58. In certain embodiments, the third cap 72 is a simple cap that may be inserted onto the second collection device 52. In other embodiments, the third cap 72 has threads 40 that may be screwed onto the second collection device 52 to seal the second open end 58. In preferred embodiments, the third cap 72 has an inner cylinder 90 taller than the third cap 72, as shown in FIG. 16. The inner cylinder 90 is configured to hold breath stabilizing media 120. For example, the breath stabilizing media 120 is a liquid, as shown in FIG. 16. In certain embodiments, the third cap 72 includes silicone material 140 surrounding the inner cylinder 90. The silicone material 140 aids in sealing in any liquid of the stabilizing media 120 and prevents leakage of the stabilizing media 120. In certain embodiments, breath stabilizing media 120 is pre-filled into the inner cylinder 90 and sealed with foil. When the third cap 72 is about to be used, the foil is removed before the third cap 72 is mounted to the second collection device 52 having microbes therein.

For the second collection device 52, a fourth cap 62 may be mounted on the second vented end 64. In certain embodiments, the fourth cap 62 is a simple cap that may be inserted onto the second collection device 52. In other embodiments, the fourth cap 62 has threads 40 that may be screwed onto the second collection device 52 to seal the second vented end 64, as shown in FIG. 17. In certain embodiments, the fourth cap 62 includes a disk of silicone material 150 inserted into the fourth cap 62. In these embodiments, a breath stabilizing media 120′, which is liquid, may be applied inside the second collection device 52 after the fourth cap 62 is mounted to the device 52. The disk of silicone material 150 aids in sealing the liquid of the stabilizing media 120′ and prevents leakage of the stabilizing media 120′.

The stabilizing media 120, 120′ and silicone materials 140 and 150 used in the first collection device 50 may be the same material as the stabilizing media 120, 120′ and silicone materials 140 and 150 used in the second collection device 52, however the same stabilizing media 120, 120′ and silicone materials 140 and 150 are not used in both devices 50, 52 when collecting a sample of breath.

An exemplary method of using a collection system is also provided. In the exemplary method, a user blows a volume of breath containing microbes into a first chamber of a breath microbe assembly. The volume of breath travels through a portion of the first chamber. The volume of breath includes upper and lower respiratory breath. When an initial volume of this breath reaches the pivoting part of the valve, the pivoting part is pushed back from the stop by this initial volume of breath and pivots in an opposite direction of the stop. This initial volume of breath, including microbes, continues to travel through the first chamber before the valve is in the activated position. When the pivoting part of the valve is pushed back, the diverting part of the valve simultaneously pivots up toward the stop and is stopped by the stop. At this time, a pathway to the second chamber is open and the volume of breath, including microbes, that followed the initial volume of breath of the user is diverted fully into the lower chamber. That is, the valve is pivoted, to direct breath containing microbes to the second collection device.

When a first collection device is mounted to the first chamber, the initial volume of breath, including microbes, that travels through the first chamber will continue to travel into the first collection device. Thus, the method includes collecting microbes of an upper respiratory breath in a first collection device.

When a second collection device is mounted to the second chamber, the following volume of breath (the volume of breath that follows the initial volume of breath), including microbes, that travels through the second chamber will continue to travel into the second collection device. Thus, the method includes collecting microbes of a lower respiratory breath in a second collection device.

To seal the collected microbes for evaluation at a later time, the method includes removing the first collection device after collecting the microbes of the upper respiratory breath, mounting a first cap on a first open end of the first collection device, mounting a second cap on a first vented end of the first collection device, removing the second collection device after collecting the microbes of the lower respiratory breath, mounting a third cap on a second open end of the second collection device, and mounting a fourth cap on a second vented end of the second collection device. The collection devices having microbes therein are now ready to be transported and/or evaluated at a later time. The collection devices may be placed in plastic bags appropriate for hazardous material transportation.

All combinations of the aforementioned embodiments throughout the entire disclosure are hereby expressly contemplated in one or more non-limiting embodiments even if such a disclosure is not described verbatim in a single paragraph or section above. In other words, an expressly contemplated embodiment may include any one or more elements described above selected and combined from any portion of the disclosure.

The subject matter of all combinations of independent and dependent claims, both singly and multiply dependent, is herein expressly contemplated. The disclosure is illustrative including words of description rather than of limitation. Many modifications and variations of the present disclosure are possible in light of the above teachings, and the disclosure may be practiced otherwise than as specifically described herein.

1. A breath microbe assembly to receive both an upper respiratory breath and a lower respiratory breath of a user, said assembly comprising:
a housing having a first chamber and a second chamber, wherein a portion of said first chamber is configured to receive said upper respiratory breath and said lower respiratory breath, and said second chamber is configured to receive said lower respiratory breath;
a stop mounted within said first chamber; and
a valve mounted to said housing and having a pivoting part and a diverting part, wherein said pivoting part is adjacent one side of said stop when said valve is in an initial position, said diverting part is adjacent another side opposite said one side of said stop when said valve is in an activated position, and said valve automatically returns to said initial position when a volume of breath of said user is dissipated.

2. The assembly of claim 1, wherein said stop is mounted at an angle within said first chamber.

3. The assembly of claim 1, further comprising a block disposed within the first chamber or the second chamber.

4. The assembly of claim 3, wherein said block is configured to prevent back flow of said upper respiratory breath into said second chamber.

5. The assembly of claim 3, wherein said block extends across a portion of said housing at an angle so that said pivoting part lays substantially flat on said block in said activated position.

6. The assembly of claim 1, wherein when a volume of breath reaches said valve, said volume of breath pushes said pivoting part to move said valve from said initial position to said activated position.

7. The assembly of claim 6, wherein said pivoting part pivots about a pivot point when pushed by said volume of breath.

8. The assembly of claim 1, further comprising a mouthpiece mounted to said housing.

9. The assembly of claim 8, wherein said mouthpiece is mounted to said first chamber.

10. The assembly of claim 1, wherein said housing is a T-shaped housing.

11. The assembly of claim 1, wherein said stop is further defined as a slanted tab that extends into said first chamber along a portion of a shape of said first chamber.

12. The assembly of claim 1, wherein said diverting part is weighted compared to said pivoting part to facilitate said automatic return of said valve.

13. The assembly of claim 1, wherein said pivoting part abuts said one side of said stop in said initial position and said diverting part abuts said another side opposite said one side of said stop in said activated position.

14. The assembly of claim 1, wherein said first chamber includes an inclined portion in said portion of said first chamber to channel a volume of breath into a smaller portion of said first chamber.

15. A collection system comprising:
a housing having a first chamber and a second chamber, wherein a portion of said first chamber is configured to receive an upper respiratory breath from a user and a lower respiratory breath from said user, and said second chamber is configured to receive said lower respiratory breath;
a valve mounted to said housing and having a pivoting part and a diverting part with the valve movable between initial and activated positions to allow a breath into a first collection device, and then to direct said breath into a second collection device when the valve is in the activated position;
said first collection device having a first open end and a first vented end and configured to collect an upper respiratory breath microbe sample from said user; and
said second collection device having a second open end and a second vented end and configured to collected a lower upper respiratory breath microbe sample from said user,
wherein said first collection device is mounted to said first chamber and wherein said second collection device is mounted to said second chamber.

16. The collection system of claim 15, wherein said first collection device is removed from said first chamber and further comprises:
a first cap mounted on said first open end and having an inner cylinder taller than said first cap, wherein said inner cylinder is configured to hold breath stabilizing media; and
a second cap mounted on said first vented end.

17. The collection system of claim 15, wherein said second collection device is removed from said second chamber and further comprises:
a third cap mounted on said second open end and having an inner cylinder taller than said third cap, wherein said inner cylinder is configured to hold breath stabilizing media; and
a fourth cap mounted on said second vented end.

18. The collection system of claim 15, wherein each of the first vented end and the second vented end include concentric circles anchored by crossed lines.

19. The collection system of claim 15, wherein said first and second collection devices are made of a material that attracts microbes.

20. A method of using a collection system, comprising:
blowing a breath containing microbes into a first chamber of a breath microbe assembly;
collecting microbes of an upper respiratory breath in a first collection device;
pivoting a valve in said breath microbe assembly to direct breath containing microbes to a second collection device;
collecting microbes of a lower respiratory breath in said second collection device;
removing said first collection device after collecting said microbes of said upper respiratory breath;
mounting a first cap on a first open end of said first collection device;
mounting a second cap on a first vented end of said first collection device;
removing said second collection device after collecting said microbes of said lower respiratory breath;
mounting a third cap on a second open end of said second collection device; and
mounting a fourth cap on a second vented end of said second collection device.
